SEQUENCE LISTING (81 sequences)

SEQ ID NO: 1 - DNA, 18 bp, Glycine max
  source 1..18: mol_type = other DNA
ccacatgtttcatattaa

SEQ ID NO: 2 - DNA, 19 bp, Glycine max
  source 1..19: mol_type = other DNA
accacatgttttatattaa

SEQ ID NO: 3 - DNA, 38 bp, Glycine max
  source 1..38: mol_type = other DNA
ctccggtttattaagaaaaattgatcataattattcgt

SEQ ID NO: 4 - DNA, 25 bp, Glycine max
  source 1..25: mol_type = other DNA
gttgggctatccacgagactatttt

SEQ ID NO: 5 - DNA, 20 bp, Glycine max
  source 1..20: mol_type = other DNA
aatcttttccagatatgcac

SEQ ID NO: 6 - DNA, 17 bp, Glycine max
  source 1..17: mol_type = other DNA
cttttccagacatgcac

SEQ ID NO: 7 - DNA, 21 bp, Glycine max
  source 1..21: mol_type = other DNA
ttgagaacccgtgcgagaatt

SEQ ID NO: 8 - DNA, 32 bp, Glycine max
  source 1..32: mol_type = other DNA
ccatctcctaagagagactttaaacaataagt

SEQ ID NO: 9 - DNA, 20 bp, Glycine max
  source 1..20: mol_type = other DNA
tcgattctcttcatattgac

SEQ ID NO: 10 - DNA, 20 bp, Glycine max
  source 1..20: mol_type = other DNA
tcgattctcttcatgttgac

SEQ ID NO: 11 - DNA, 30 bp, Glycine max
  source 1..30: mol_type = other DNA
gctcttgcttcttctaaactaacttcaagt

SEQ ID NO: 12 - DNA, 22 bp, Glycine max
  source 1..22: mol_type = other DNA
gactatcaccctcgacgatgtg

SEQ ID NO: 13 - DNA, 17 bp, Glycine max
  source 1..17: mol_type = other DNA
catgacccactgttcta

SEQ ID NO: 14 - DNA, 17 bp, Glycine max
  source 1..17: mol_type = other DNA
catgacccattgttcta

SEQ ID NO: 15 - DNA, 25 bp, Glycine max
  source 1..25: mol_type = other DNA
ccaccatggttcttcagatttgtct

SEQ ID NO: 16 - DNA, 23 bp, Glycine max
  source 1..23: mol_type = other DNA
gcttcaaccatggtggacagaaa

SEQ ID NO: 17 - DNA, 16 bp, Glycine max
  source 1..16: mol_type = other DNA
actcgttacggatttg

SEQ ID NO: 18 - DNA, 16 bp, Glycine max
  source 1..16: mol_type = other DNA
actcgttacagatttg

SEQ ID NO: 19 - DNA, 29 bp, Glycine max
  source 1..29: mol_type = other DNA
cttgaaaattgtcctttggtgaatctgtt

SEQ ID NO: 20 - DNA, 38 bp, Glycine max
  source 1..38: mol_type = other DNA
gtagcaaataaaaaattatcactattgaccgatatgaa

SEQ ID NO: 21 - DNA, 19 bp, Glycine max
  source 1..19: mol_type = other DNA
ttgtcgaacataactttca

SEQ ID NO: 22 - DNA, 16 bp, Glycine max
  source 1..16: mol_type = other DNA
tcgaacatagctttca

SEQ ID NO: 23 - DNA, 37 bp, Glycine max
  source 1..37: mol_type = other DNA
tcgttaaatagtacagttatctttttttttcaatgca

SEQ ID NO: 24 - DNA, 25 bp, Glycine max
  source 1..25: mol_type = other DNA
cctccccgtgtaagtagaaatcatt

SEQ ID NO: 25 - DNA, 17 bp, Glycine max
  source 1..17: mol_type = other DNA
cctatgcaatcaattta

SEQ ID NO: 26 - DNA, 16 bp, Glycine max
  source 1..16: mol_type = other DNA
ctatgcagtcaattta

SEQ ID NO: 27 - DNA, 24 bp, Glycine max
  source 1..24: mol_type = other DNA
acaaccatccagtgttgttttcct

SEQ ID NO: 28 - DNA, 24 bp, Glycine max
  source 1..24: mol_type = other DNA
agaaggtgctacatgctagtgttg

SEQ ID NO: 29 - DNA, 18 bp, Glycine max
  source 1..18: mol_type = other DNA
aggtgacttagtggaatc

SEQ ID NO: 30 - DNA, 18 bp, Glycine max
  source 1..18: mol_type = other DNA
aggtgacttaatggaatc

SEQ ID NO: 31 - DNA, 29 bp, Glycine max
  source 1..29: mol_type = other DNA
tttttggcatttttacgaacacatagagt

SEQ ID NO: 32 - DNA, 30 bp, Glycine max
  source 1..30: mol_type = other DNA
tgagctttgaactagatgaataacctttgt

SEQ ID NO: 33 - DNA, 20 bp, Glycine max
  source 1..20: mol_type = other DNA
aagaccaatggtatgacaag

SEQ ID NO: 34 - DNA, 17 bp, Glycine max
  source 1..17: mol_type = other DNA
accaatggtgtgacaag

SEQ ID NO: 35 - DNA, 27 bp, Glycine max
  source 1..27: mol_type = other DNA
cacgtacctctgataggaaatcattga

SEQ ID NO: 36 - DNA, 31 bp, Glycine max
  source 1..31: mol_type = other DNA
ttcttattgtcgtcctcttctttgttaatgt

SEQ ID NO: 37 - DNA, 18 bp, Glycine max
  source 1..18: mol_type = other DNA
acaagacaaaggatttga

SEQ ID NO: 38 - DNA, 17 bp, Glycine max
  source 1..17: mol_type = other DNA
caagacaaagaatttga

SEQ ID NO: 39 - DNA, 29 bp, Glycine max
  source 1..29: mol_type = other DNA
ggaggccaaatttaaaggtttttcatttc

SEQ ID NO: 40 - DNA, 30 bp, Glycine max
  source 1..30: mol_type = other DNA
ttacaatgattcccatccatcataaacatt

SEQ ID NO: 41 - DNA, 23 bp, Glycine max
  source 1..23: mol_type = other DNA
agttatactgaacttaaatactt

SEQ ID NO: 42 - DNA, 19 bp, Glycine max
  source 1..19: mol_type = other DNA
atactgaacttgaatactt

SEQ ID NO: 43 - DNA, 35 bp, Glycine max
  source 1..35: mol_type = other DNA
tgtatttttggttcataaaatttaaatttgagatc

SEQ ID NO: 44 - DNA, 32 bp, Glycine max
  source 1..32: mol_type = other DNA
accttccttttttccccctaaattaaacatat

SEQ ID NO: 45 - DNA, 19 bp, Glycine max
  source 1..19: mol_type = other DNA
agtatgcttgccgcaaaac

SEQ ID NO: 46 - DNA, 24 bp, Glycine max
  source 1..24: mol_type = other DNA
gcatcaaccaagccttatgcatat

SEQ ID NO: 47 - DNA, 24 bp, Glycine max
  source 1..24: mol_type = other DNA
tgtttatggctctctccaaagtca

SEQ ID NO: 48 - DNA, 20 bp, Glycine max
  source 1..20: mol_type = other DNA
tgcttgccgcaaaattagag

SEQ ID NO: 49 - DNA, 24 bp, Glycine max
  source 1..24: mol_type = other DNA
gcatcaaccaagccttatgcatat

SEQ ID NO: 50 - DNA, 21 bp, Glycine max
  source 1..21: mol_type = other DNA
tctctacatgcccaaaccacc

SEQ ID NO: 51 - DNA, 6809 bp, Glycine max
  source 1..6809: mol_type = genomic DNA
tagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggtcttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagaaatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatatctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaattagagagagggttggagtagcgcctattgtagagaagatggtggaaaatagacttaggtggtttgggcatgtagagagaagaccggtagactctgtagtgaggagagtagaccagatggagagaagacaaacaattcgaggcagaggaagacccaaaaagactataagagaggttataaaaaaggatctcgaaattaatggtttggatagaagtatggtacttgatagaacattatggcggaagttgatccatgtagccgaccccacctagtgggataaggcgttgttgttgttgttgttgttgtatgcttgccgcaaaactttggcggatagccggccccgggttagaggaaggtttgcaaagaatgatgactttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttataaatatttaggaacttgcttttaaggttttttttttttt

SEQ ID NO: 52 - DNA, 6527 bp, Glycine max
  source 1..6527: mol_type = genomic DNA
tagacccctgtaggccggcttagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggtcttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagaaatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatgtctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaactttggcggatagccggccccgggttagaggaaggtttgcaaagaatgatgactttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttataaatatttaggaacttgcttttaaggtttttttttttttttg

SEQ ID NO: 53 - DNA, 8770 bp, Glycine max
  source 1..8770: mol_type = genomic DNA
tagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggtcttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagaaatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatatctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaattagagagagggttggagtagcgcctattgtagagaagatggtggaaaatagacttaggtggtttgggcatgtagagagaagaccggtagactctgtagtgaggagagtagaccagatggagagaagacaaacaattcgaggcagaggaagacccaaaaagactataagagaggttataaaaaaggatctcgaaattaatggtttggatagaagtatggtacttgatagaacattatggcggaagttgatccatgtagccgaccccacctagtgggataaggcgttgttgttgttgttgttgttgtatgcttgccgcaaaactttggcggatagccggccccgggttagaggaaggtttgcaaagaatgatgactttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttataaatatttaggaacttgcttttaaggttttttttttttttgatgatgggttgcttttaagtaattttacatcctctaattatttttttcttaaatatgggattaaattgattgttacttgttgaagctaaaaaaggtttataatgttatggactaaattgatgttgtattgatttattggttcaactaaaataagaatataatggtaacacaataataatatcatttactcgtaaattattcttggtataatttttaaaatgattattataaaaatcaacaaaattattatatatgatgagttataattagatgaggtatatattttacaccgtgaatgtttccttattttcttaaaaataaaatgatggtaaaccttaaatcctatagtagcgctaaactaggttaagcttgcaactcttattcgctaacctggtgacaacagaactcttttgtttggacatttgcctagtaaagattagaagaggtccacaatggatggaaaggtacagttatacttctatttcggtaacttttagaatatttggcaaaattctcactaaacttgtagaatactttattcgttaaatagtacagttatctttttttttcaatgcaaaataatttaattgtcgaacataactttcaagagataaatgatttctacttacacggggaggataattgaatgtgggatttttttttattttacttctttagttctttatgggaaagaacttttaattaattcagaattcgatcataatttcgttaaagatcaaatatcaaatgattcaatcttaattttaatacattaattatttattataacgtgatttgatctcatattttttctatggtcaataaaatattggctaaatgatacgtgtagtcttttatgttattgtttagatttaatttaattatttatcttttaaatttagtttcatttaatcattctgcccgtttaaaattaatgttgttaataattaacatatcgacaattaacaatcatcataaaataatatttaacacgtaataatcacagtgataatttttaaccacataatagtaattaaaaattattaaaaaccctttccaaaagaactttacaatttttgtggtcgatttttctcgtacaatttcatgaatcttaagattgatttaggggttttgttacccatgtgtttcaatcaacatagtcaaaaatcaaataattttgttgaagcaagtgtacgaataagatatttcatgtaaattttactaaatttagtttgttttgatttatgctttgagtttatgttttctgcaagcaacattttcaaaaattgtttaacggattggtctcacgcaagcaacattttcaaaaattgtttaacggattggtctcacgcaagcaacatttaaaaaaaaatcatttcgatgaagaatcaaagagcacgaattgggtcaagttataggttggagaaacctattttctatagcatgagtttaacaatacttgctaaattcgttcaatctatgcatccatttgttgcagaatttaggcagttttgattggaatgcatcaatctggagtctccctcaatcaattttaagtttcagcctgtcagggaagtatatgagaaatcgaatatgaaaattttaaagttattagcagacagatttttaattttttataatttttaaatattatcgtgtcagttaaaaattgtcattatgttaattacatgtgcataaatacatccaaaataaaaattattatttgacttaaatatttttttagttttccaaatataaggtattcctattttagtccctcaaattaaaaattatttttccagtcctacaaaatcatatttgtagtccttatccgtatatgcattagtgggtgtcaaacgaccaacattaaattcacacttcgtgacaacttttaaccgtaacatttgatttttaatacaaattttaaaccaccgtgatttctaggggttaaaacctcca

SEQ ID NO: 54 - DNA, 6823 bp, Glycine max
  source 1..6823: mol_type = genomic DNA
ctctgtaggccggcctagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggtcttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagaaatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatatctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaattagagagagggttggagtagcgcctattgtagagaagatggtggaaaatagacttaggtggtttgggcatgtagagagaagaccggtagactctgtagtgaggagagtagaccagatggagagaagacaaacaattcgaggcagaggaagacccaaaaagactataagagaggttataaaaaaggatctcgaaattaatggtttggatagaagtatggtacttgatagaacattatggcggaagttgatccatgtagccgaccccacctagtgggataaggcgttgttgttgttgttgttgttgtatgcttgccgcaaaactttggcggatagccggccccgggttagaggaaggtttgcaaagaatgatgactttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttataaatatttaggaacttgcttttaaggtttttttttttt

SEQ ID NO: 55 - DNA, 6809 bp, Glycine max; identical to SEQ ID NO: 51
  source 1..6809: mol_type = genomic DNA

SEQ ID NO: 56 - DNA, 6939 bp, Glycine max
  source 1..6939: mol_type = genomic DNA
aggccaagtcgtagacccctgtaggccggcttagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggtcttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagatatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatgtctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacaacaacaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaactttggcggatagccggccccgggttagaggaaggtttgcaaagaatgatgattttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttataaatatttaggaacttgcttttaaggtttttttttttttttgatgatgggttgcttttaagtaattttacatcctctaattatttttttcttaaatatgggattaaattgattgttacttgttgaagctaaaaaaggtttataatgttatggactaaattgatgttgtattgatttattggttcaactaaaataagaatataatggtaacacaataataatatcatttactcgtaaattatttttggtataatttttaaaatgattattataaaaatcaacaaaattattatatatgatgagttataattagatgaggtatatattttacaccgtgaatgtttccttattttcttaaaaataaaatgatggtaaaccttaaatcctatagtagcgctaaactaggttaagcttgcaactcttattcgctaacctggtg

SEQ ID NO: 57 - DNA, 6527 bp, Glycine max; identical to SEQ ID NO: 52
  source 1..6527: mol_type = genomic DNA

SEQ ID NO: 58 - DNA, 4794 bp, Glycine soja
  source 1..4794: mol_type = genomic DNA
tctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatgtctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaggaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcatcctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaactttggcggatagccggccccgggttagaggaaggtttgcaaagaatgatgactttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttataaatatttaggaacttgcttttaaggtttttttttttttttggagggggggggcttttaaaaaattttcccccccccaattttttttttttaaaaaaggggaaaaa

SEQ ID NO: 59 - DNA, 321 bp, Glycine max
  source 1..321: mol_type = genomic DNA
ttagagagagggttggagtagcgcctattgtagagaagatggtggaaaatagacttaggtggtttgggcatgtagagagaagaccggtagactctgtagtgaggagagtagaccagatggagagaagacaaacaattcgaggcagaggaagacccaaaaagactataagagaggttataaaaaaggatctcgaaattaatggtttggatagaagtatggtacttgatagaacattatggcggaagttgatccatgtagccgaccccacctagtgggataaggcgttgttgttgttgttgttgttgtatgcttgccgcaaaa

SEQ ID NO: 60 - DNA, 401 bp, Glycine max
  source 1..401: mol_type = genomic DNA
gcttgagtcattaatttcctttttcatctagatcttcgactacttaattagtctttggggccttgaaatatccatcttcatcttaagagattcacacttgtgttaattgattttgggttacctattttttttttatattaatcctccggtttattaagaaaaattgatcataattattcgtataagtatgtatttaatatgaaacatgtggttggttttaaaatagtctcgtggatagcccaacaaatacatattcattgaaatattcatgccccaaacattactagttttgttcagtctcaaaaaataaataaagtctaatcataaattatttctgttaatgattaaactcagatagtactaaaacgatttaattttaacttaatcaattgtgtctaatg

SEQ ID NO: 61 - DNA, 401 bp, Glycine max; identical to SEQ ID NO: 60
  source 1..401: mol_type = genomic DNA

SEQ ID NO: 62 - DNA, 401 bp, Glycine max
  source 1..401: mol_type = genomic DNA
tcagtgtgtgagaaatggaaaattcatccgaagcctaataagtatttatgtgaattctcatgtgaattgcaaaagtacgaatcatacaaaagcctataatgttgccgtgtaccaaagattaacttattgataacaaagaatcacacgaatagtgttatattatacaatattattgagaacccgtgcgagaattagtgcatatctggaaaagattaatttcttcaaaaataatcacttattgtttaaagtctctcttaggagatgggaaaaataaacaattgtttctctaaaagactaaaacaagttggaccaaacatgcacttagacattagaagtttagaactgctaaccaattaatctgtaacctcatggtacattaaattggaaatcgagttaatggc

SEQ ID NO: 63 - DNA, 401 bp, Glycine max
  source 1..401: mol_type = genomic DNA
tcagtgtgtgagaaatggaaaattcatccgaagcctaataagtatttatgtgaattctcatgtgaattgcaaaagtacgaatcatacaaaagcctataatgttgccgtgtaccaaagattaacttattgataacaaagaatcacacgaatagtgttatattatacaatattattgagaacccgtgcgagaattagtgcatgtctggaaaagattaatttcttcaaaaataatcacttattgtttaaagtctctcttaggagatgggaaaaataaacaattgtttctctaaaagactaaaacaagttggaccaaacatgcacttagacattagaagtttagaactgctaaccaattaatctgtaacctcatggtacattaaattggaaatcgagttaatggc

SEQ ID NO: 64 - DNA, 401 bp, Glycine max
  source 1..401: mol_type = genomic DNA
agcaaagagtgtgcaacctaccaaatgcaacaaatatgatcaagttgctacaatccactatgttgtgtcacatttgctccaatatatgtctcgcagctaggatagcctaacaaattccccatggcattgtactgtttcagctcttgcttcttctaaactaacttcaagtaattcaactaatagtaagatggcttcgtcaatatgaagagaatcgaagttgcggaaggcccctataatcgaaaaatgtagcaacgatgccacatcgtcgagggtgatagtcacctctcctacaggaagatggaaactactagtttccttatgccaccacttttcaaatactaacataagtctccgatcaccagtgtccaacaaacatgcgatcaaaggacttaatcctgtgg

SEQ ID NO: 65 - DNA, 401 bp, Glycine max
  source 1..401: mol_type = genomic DNA
agcaaagagtgtgcaacctaccaaatgcaacaaatatgatcaagttgctacaatccactatgttgtgtcacatttgctccaatatatgtctcgcagctaggatagcctaacaaattccccatggcattgtactgtttcagctcttgcttcttctaaactaacttcaagtaattcaactaatagtaagatggcttcgtcaacatgaagagaatcgaagttgcggaaggcccctataatcgaaaaatgtagcaacgatgccacatcgtcgagggtgatagtcacctctcctacaggaagatggaaactactagtttccttatgccaccacttttcaaatactaacataagtctccgatcaccagtgtccaacaaacatgcgatcaaaggacttaatcctgtgg

SEQ ID NO: 66 - DNA, 401 bp, Glycine max
  source 1..401: mol_type = genomic DNA
agaaggatcttctgtaggtattttggttctaacaatgttcggtcgattaggaaaatagcctccataagtatattgggtaaagttaacagctgcatgatgagcagatgctgtccaagctatggtggtgatgatgtcaataaggtcttttggtgttttgagatttggccaccatggttcttcagatttgtctccatgacccactgttctaatttctgtccaccatggttgaagctcttggtcagattctatgatacttggggttggatagtaatggttgacatactcagttatccattgtttgatggcatcccatatgagaagaccatcattggcaaaagggtagtcttcaattgttagctttaggccatgtggtgcatttggatctaccacagcaattcctc

SEQ ID NO: 67 - DNA, 401 bp, Glycine max
  source 1..401: mol_type = genomic DNA
agaaggatcttctgtaggtattttggttctaacaatgttcggtcgattaggaaaatagcctccataagtatattgggtaaagttaacagctgcatgatgagcagatgctgtccaagctatggtggtgatgatgtcaataaggtcttttggtgttttgagatttggccaccatggttcttcagatttgtctccatgacccattgttctaatttctgtccaccatggttgaagctcttggtcagattctatgatacttggggttggatagtaatggttgacatactcagttatccattgtttgatggcatcccatatgagaagaccatcattggcaaaagggtagtcttcaattgttagctttaggccatgtggtgcatttggatctaccacagcaattcctc

SEQ ID NO: 68 - DNA, 401 bp, Glycine max
  source 1..401: mol_type = genomic DNA
taagaaggcaaggaaagctttggaaatgtaaagcatgaactgaaaccatgtttgcggtttttaaaatgaatgatatttctacaatgccacctttgcttcctgcacccaccaatatacttgtacacctacaccatttcggataacttgaaaattgtcctttggtgaatctgttaagtattacaaatacataaactcgttacggatttgaaattcatatcggtcaatagtgataattttttatttgctactattttgagtgtgtactgtggaagcaatgttttccaagtttattttgatgatgacaaagactcaagtcaagaatcaagaatcaaagcaagtttcaagaatcaaagagtcattcaatcaaagcaaggttcaagaatcaagattaaagtgaagat

SEQ ID NO: 69 - DNA, 401 bp, Glycine max
  source 1..401: mol_type = genomic DNA
taagaaggcaaggaaagctttggaaatgtaaagcatgaactgaaaccatgtttgcggtttttaaaatgaatgatatttctacaatgccacctttgcttcctgcacccaccaatatacttgtacacctacaccatttcggataacttgaaaattgtcctttggtgaatctgttaagtattacaaatacataaactcgttacagatttgaaattcatatcggtcaatagtgataattttttatttgctactattttgagtgtgtactgtggaagcaatgttttccaagtttattttgatgatgacaaagactcaagtcaagaatcaagaatcaaagcaagtttcaagaatcaaagagtcattcaatcaaagcaaggttcaagaatcaagattaaagtgaagat

SEQ ID NO: 70 - DNA, 401 bp, Glycine max
  source 1..401: mol_type = genomic DNA
acagaactcttttgtttggacatttgcctagtaaagattagaagaggtccacaatggatggaaaggtacagttatacttctatttcggtaacttttagaatatttggcaaaattctcactaaacttgtagaatactttattcgttaaatagtacagttatctttttttttcaatgcaaaataatttaattgtcgaacataactttcaagagataaatgatttctacttacacggggaggataattgaatgtgggatttttttttattttacttctttagttctttatgggaaagaacttttaattaattcagaattcgatcataatttcgttaaagatcaaatatcaaatgattcaatcttaattttaatacattaattatttattataacgtgatttgat

SEQ ID NO: 71 - DNA, 401 bp, Glycine max
  source 1..401: mol_type = genomic DNA
acagaactcttttgtttggacatttgcctagtaaagattagaagaggtccacaatggatggaaaggtacagttatacttctatttcggtaacttttagaatatttggcaaaattctcactaaacttgtagaatactttattcgttaaatagtacagttatctttttttttcaatgcaaaataatttaattgtcgaacatagctttcaagagataaatgatttctacttacacggggaggataattgaatgtgggatttttttttattttacttctttagttctttatgggaaagaacttttaattaattcagaattcgatcataatttcgttaaagatcaaatatcaaatgattcaatcttaattttaatacattaattatttattataacgtgatttgat

SEQ ID NO: 72 - DNA, 401 bp, Glycine max
  source 1..401: mol_type = genomic DNA
atatcatgttaatgatctccttttgtcacgatctccggcaaaatatgcatccacatagccttttgctccacctgaattttgactatcttttccatatttcaaacttgtattaacaaacccctttaggtatcttaaaatctcttttacaaccatccagtgttgttttcctagatccttcataaaccgactgactaaattgattgcataggcgatgtcttggtgagtacacactatagcatacattagactgtctgcaacactagcatgtagcaccttctccatttcatcgacttcttgagtagtttttggacagtcttctagatataatttgaagtgagctggaacaaggatgcttactagtttagcctccttcatctaggacctgtttaaaaccttctcaa

SEQ ID NO: 73 - DNA, 401 bp, Glycine max
  source 1..401: mol_type = genomic DNA
atatcatgttaatgatctccttttgtcacgatctccggcaaaatatgcatccacatagccttttgctccacctgaattttgactatcttttccatatttcaaacttgtattaacaaacccctttaggtatcttaaaatctcttttacaaccatccagtgttgttttcctagatccttcataaaccgactgactaaattgactgcataggcgatgtcttggtgagtacacactatagcatacattagactgtctgcaacactagcatgtagcaccttctccatttcatcgacttcttgagtagtttttggacagtcttctagatataatttgaagtgagctggaacaaggatgcttactagtttagcctccttcatctaggacctgtttaaaaccttctcaa

SEQ ID NO: 74 - DNA, 401 bp, Glycine max
  source 1..401: mol_type = genomic DNA
tatataaaatagtataagatgtagaagcaaagttagagttaataaataaataaaacaataataaatttaattggattgtgaaaatgtaaataaaaatagcataaataaataaaaaattgtttttggcatttttacgaacacatagagtgcgataaaaagatgtaaaataacaaagaataaaagagttgaggttgattccactaagtcacctcctaatgcaaacaaaggttattcatctagttcaaagctcaattattattaacaactcactaaaatactaaaaccctgatcactcaatgggaaaaatctaagcttcttgaccaaactactaatcccttgactaatctaattaagtgacttgttttaagaacaagagttaaaagacgacaaataagccttct

SEQ ID NO: 75 - DNA, 401 bp, Glycine max
  source 1..401: mol_type = genomic DNA
tatataaaatagtataagatgtagaagcaaagttagagttaataaataaataaaacaataataaatttaattggattgtgaaaatgtaaataaaaatagcataaataaataaaaaattgtttttggcatttttacgaacacatagagtgcgataaaaagatgtaaaataacaaagaataaaagagttgaggttgattccattaagtcacctcctaatgcaaacaaaggttattcatctagttcaaagctcaattattattaacaactcactaaaatactaaaaccctgatcactcaatgggaaaaatctaagcttcttgaccaaactactaatcccttgactaatctaattaagtgacttgttttaagaacaagagttaaaagacgacaaataagccttct

SEQ ID NO: 76 - DNA, 401 bp, Glycine max
  source 1..401: mol_type = genomic DNA
aatgatacattttcatctagttacataagatatcaaaataatctagtaaatgtaattaagaatattctactaaacttgtcacaaaaattttctctaaacactttaggaaacacttttcattgtcaatctacacgtacctctgataggaaatcattgaagaaggcattttaaccatcgttggatgatcctattacttgtcataccattggtctttacattaacaaagaagaggacgacaataagaatttgttgtggcctttaatcttgtacacaaaaggcatttacagtatactttattttgctattggagtctagccatttaaaggcatatttaaggttgtgtaaaagacctgaatatggaactgctcttagtatgaatgatggtttgacaggaggatgct

SEQ ID NO: 77 - DNA, 401 bp, Glycine max
  source 1..401: mol_type = genomic DNA
aatgatacattttcatctagttacataagatatcaaaataatctagtaaatgtaattaagaatattctactaaacttgtcacaaaaattttctctaaacactttaggaaacacttttcattgtcaatctacacgtacctctgataggaaatcattgaagaaggcattttaaccatcgttggatgatcctattacttgtcacaccattggtctttacattaacaaagaagaggacgacaataagaatttgttgtggcctttaatcttgtacacaaaaggcatttacagtatactttattttgctattggagtctagccatttaaaggcatatttaaggttgtgtaaaagacctgaatatggaactgctcttagtatgaatgatggtttgacaggaggatgct

SEQ ID NO: 78 - DNA, 401 bp, Glycine max
  source 1..401: mol_type = genomic DNA
attgttgatgaatcaagtttggaaaattgtatggtgcgccagggtatgggccttatggtgccatagaaacaacataatttttcgccatcaattacttgacttggaatctatcatggaggtaataaaatataagttttgggcatggttggaggccaaatttaaaggtttttcatttctcttattttgagtggtattcaaatcctttgtcttgtatacaatgtttatgatggatgggaatcattgtaatggggagggaaatgagatcagaaggcttaagtcgggtctaaaatggtgctagctagatgaatatcaacatgtgatatgcgatatggtgaatgtatgtgcagttacgtttaccggtaccactggtaaccttataaataaatctcttttggctgatg

SEQ ID NO: 79 - DNA, 401 bp, Glycine max
  source 1..401: mol_type = genomic DNA
attgttgatgaatcaagtttggaaaattgtatggtgcgccagggtatgggccttatggtgccatagaaacaacataatttttcgccatcaattacttgacttggaatctatcatggaggtaataaaatataagttttgggcatggttggaggccaaatttaaaggtttttcatttctcttattttgagtggtattcaaattctttgtcttgtatacaatgtttatgatggatgggaatcattgtaatggggagggaaatgagatcagaaggcttaagtcgggtctaaaatggtgctagctagatgaatatcaacatgtgatatgcgatatggtgaatgtatgtgcagttacgtttaccggtaccactggtaaccttataaataaatctcttttggctgatg

SEQ ID NO: 80 - DNA, 401 bp, Glycine max
  source 1..401: mol_type = genomic DNA
aacaaatcggtaatagaagcaatctagaaaatcaaaaggaacacaaccattcaagagaagataattataactcaattcatctcctaggccttgctcttccattaccatgatagttttttttttttatgttatcattggtacataatatttatgtatttttggttcataaaatttaaatttgagatctaatttaaagtatttaagttcagtataactcaaccaatgacttataattatcataaatttttatatatgtttaatttagggggaaaaaaggaaggttaagcagaaaagattaccgaatcctctccaaatttaatccaaaagcgagccaaggccctttgatggttgtcttttggcagcaatgggtggttctctggccacgtttcttcgatgtattc

SEQ ID NO: 81 - DNA, 401 bp, Glycine max
  source 1..401: mol_type = genomic DNA
aacaaatcggtaatagaagcaatctagaaaatcaaaaggaacacaaccattcaagagaagataattataactcaattcatctcctaggccttgctcttccattaccatgatagttttttttttttatgttatcattggtacataatatttatgtatttttggttcataaaatttaaatttgagatctaatttaaagtattcaagttcagtataactcaaccaatgacttataattatcataaatttttatatatgtttaatttagggggaaaaaaggaaggttaagcagaaaagattaccgaatcctctccaaatttaatccaaaagcgagccaaggccctttgatggttgtcttttggcagcaatgggtggttctctggccacgtttcttcgatgtattc

(19) United States
(12) Patent Application Publication
(10) Pub. No.: US 2026/0107892 A1
(43) Pub. Date: Apr. 23, 2026

(54) METHODS FOR PRODUCING HIGH PROTEIN SOYBEANS

(71) Applicant: PIONEER HI-BRED INTERNATIONAL, INC., JOHNSTON, IA (US)

(72) Inventors: KRISTIN HAUG COLLET, DES MOINES, IA (US); NICHOLE HUITT, PERRY, IA (US); SIVA S. AMMIRAJU JETTY, JOHNSTON, IA (US); BO SHEN, JOHNSTON, IA (US); YANG WANG, JOHNSTON, IA (US)

(73) Assignee: PIONEER HI-BRED INTERNATIONAL, INC., JOHNSTON, IA (US)

(21) Appl. No.: 19/117,729

(22) PCT Filed: Oct. 2, 2023
(86) PCT No.: PCT/US2023/075685

§ 371 Date: Apr. 2, 2025

Related U.S. Application Data
(60) Provisional application No. 63/378,147, filed on Oct. 3, 2022.

(51) Int. Cl. A01H 1/00; C12Q 1/6895

(52) U.S. Cl. CPC A01H 1/108; C12Q 1/6895; C12Q 2600/13

(57) ABSTRACT

The present disclosure provides methods and compositions for producing, detecting, and selecting soybean plants and seeds comprising at least one high protein CCT (CONSTANS, CO-like and TOC1) domain containing variant allele and introgressing the high protein CCT variant allele into soybean plants. The present disclosure also provides methods and compositions for producing, detecting, and selecting soybean plants producing seeds having a high protein content including breeding methods for introgressing high protein alleles into soybean plants using marker assisted selection using markers linked to or associated with high protein CCT in soybean.

### REFERENCE TO SEQUENCE LISTING SUBMITTED ELECTRONICALLY

The official copy of the sequence listing is submitted electronically via Patent Center as an XML formatted sequence listing with a file named 108282_SequenceListing created on Sep. 19, 2023, and having a size of 134,402 bytes and is filed concurrently with the specification. The sequence listing comprised in this XML formatted document is part of the specification and is herein incorporated by reference in its entirety.

### BACKGROUND

Soybeans are a major agriculture commodity in many parts of the world, and are a source of useful products, such as protein and oil, for human and animal consumption. A valuable product obtained from processed soybeans is soybean meal, which contains a high proportion of protein and is primarily used as a component in animal feed. Soy meal can be further processed to produce soy protein isolates, soy flour or soy concentrates, which can be used in foods, glues and as emulsifiers and texturizers. Soybean plants which produce seeds higher in protein content may contribute to a higher-value crop.

### SUMMARY

Provided herein are methods for producing plants having a high protein CCT allele comprising isolating one or more nucleic acids from a soybean population comprising a plurality of soybean plants in which the soybean plants comprise a CCT gene and the soybean population comprises a high protein CCT allele of the CCT gene and a wild-type CCT allele of the CCT gene, assaying the nucleic acids for the presence of the high protein CCT allele by detecting a nucleotide polymorphism in the CCT gene sequence having at least 95% identity to SEQ ID NO: 51, assaying the one or more nucleic acids for the presence of the wild-type CCT allele having at least 95% identity to SEQ ID NO: 51, selecting from the plurality of soybean plants one or more soybean plants comprising two high protein CCT alleles or comprising one high protein CCT allele and one wild-type CCT allele, or a combination thereof, and crossing the selected soybean plants with a second soybean plant, or self-pollinating the selected plants, to produce a plant having the high-protein CCT allele. In certain embodiments, the plant selected is homozygous for the high protein CCT allele. In certain embodiments, the method further comprises detecting in the one or more nucleic acids at least one marker locus associated with high protein seeds located within a chromosome interval flanked by and including marker locus S20007K-001-Q001 and marker locus S20008A-001-Q001.

Also provided are methods for selecting plants in a segregating population having a high protein CCT allele comprising self-pollinating a first soybean plant or first soybean germplasm or crossing the first soybean plant or first soybean germplasm with a second soybean plant or second soybean germplasm to form a soybean population comprising a plurality of soybean plants or soybean germplasm, the soybean plants or soybean germplasm comprising a CCT gene and the soybean population comprising a high protein CCT allele of the CCT gene and a wild-type CCT allele of the CCT gene, isolating nucleic acids from the soybean plants or soybean germplasm of the population, assaying the one or more nucleic acids for the presence of the high protein CCT allele by detecting a nucleotide polymorphism in the CCT gene sequence having at least 95% identity to SEQ ID NO: 51, assaying the one or more nucleic acids for the presence of the wild-type CCT allele having at least 95% identity to SEQ ID NO: 51, and selecting from the plurality of soybean plants or soybean germplasm one or more soybean plants or soybean germplasm comprising two high protein CCT alleles or comprising one high protein CCT allele and one wild-type CCT allele, or a combination thereof. In certain embodiments, the plant selected is homozygous for the high protein CCT allele. In certain embodiments, the method further comprises detecting in the one or more nucleic acids at least one marker locus associated with high protein seeds located within a chromosome interval flanked by and including marker locus S20007K-001-Q001 and marker locus S20008A-001-Q001.

Further provided are methods for introgressing a high protein CCT domain containing variant sequence into a soybean plant or soybean germplasm comprising crossing a first soybean plant or first soybean germplasm with a second soybean plant or second soybean germplasm to form a soybean plant or soybean germplasm population, wherein the first soybean plant or soybean germplasm or the second soybean plant or germplasm comprises the high protein CCT domain containing variant sequence, isolating nucleic acids from the soybean plants or soybean germplasm of the population, assaying the one or more nucleic acids for the presence of the high protein CCT allele by detecting a nucleotide polymorphism in the CCT gene sequence having at least 95% identity to SEQ ID NO: 51, assaying the one or more nucleic acids for the presence of a wild-type CCT allele having at least 95% identity to SEQ ID NO: 51, and selecting from the plurality of soybean plants or soybean germplasm one or more soybean plants or soybean germplasm comprising at least one high protein CCT allele. In certain embodiments, the plant selected is homozygous for the high protein CCT allele. In certain embodiments, the method further comprises detecting in the one or more nucleic acids at least one marker locus associated with high protein seeds located within a chromosome interval flanked by and including marker locus S20007K-001-Q001 and marker locus S20008A-001-Q001.

In certain embodiments of the methods described herein, the nucleotide polymorphism is a deletion. In certain embodiments, the deletion comprises at least 10, 25, 50, 75, 100, 125, 150, 175, 200, 225, 250, 275, 300, or 325 nucleotides. In certain embodiments, the deletion has at least 95% sequence identity to SEQ ID NO: 59. In certain embodiments, the deletion is detected using a probe sequence, such as, for example, the sequence of SEQ ID NO: 45. In certain embodiments of the methods described herein, the polymorphism comprises a single nucleotide polymorphism (SNP). In certain embodiments, the SNP is nucleotide G at marker locus S200081-001-Q001.

In certain embodiments of the methods described herein, the wild-type CCT allele is detected using an assay that detects the presence of a nucleotide sequence having at least 95% sequence identity to SEQ ID NO: 59. In certain embodiments, the wild-type CCT allele is detected using a nucleotide probe that selectively hybridizes to a fragment of the nucleotide sequence having at least 95% sequence identity to SEQ ID NO: 59, such as, for example SEQ ID NO: 48.

In certain embodiments of the methods described herein, the assays for detecting the presence of the high protein CCT allele and the wild-type CCT allele occurs in the same reaction vessel. In certain embodiments of the methods described herein, the assays for detecting the presence of the high protein CCT allele and the wild-type CCT allele occurs simultaneously, optionally, in the same reaction vessel.

Provided are methods for producing a soybean plant producing high protein seeds comprising isolating one or more nucleic acids from a soybean population comprising a plurality of soybean plants, and detecting in the one or more nucleic acids at least one marker locus associated with high protein seeds located within a chromosome interval flanked by and including marker locus S20007K-001-Q001 and marker locus S20008A-001-Q001, wherein the chromosomal interval comprises a G at marker locus S200081-001-Q001. In certain embodiments, the method further comprises selecting a plant comprising the at least one marker locus associated with high protein seeds. In certain embodiments, the method further comprises crossing the selected plant with a second soybean plant.

Also provided are methods for producing a soybean plant or soybean germplasm having high protein seeds comprising crossing a first soybean plant or first soybean germplasm with a second soybean plant or second soybean germplasm to form a soybean plant or soybean germplasm population, isolating nucleic acids from the soybean plants or soybean germplasm of the population, detecting in the nucleic acids at least one marker locus associated with high protein seeds located within a chromosome interval flanked by and including marker locus S20007K-001-Q001 and marker locus S20008A-001-Q001, wherein the chromosomal interval comprises a G at marker locus S200081-001-Q001, and selecting, if present, one or more soybean plants or soybean germplasm of the population comprising the detected marker locus.

In certain embodiments of the methods described herein, the marker locus located within the chromosome interval flanked by and including marker locus S20007K-001-Q001 and marker locus S20008A-001-Q001 associated with high protein seeds comprises marker locus S20007K-001-Q001, S20007N-001-Q001, S20007R-001-Q001, S20007T-001-Q001, S20007W-001-Q001, S200099-00-Q001, S200081-001-Q001, S200083-001-Q001, S200085-001-Q001, S200086-001-Q001, S200093-001-Q001, and S20008A-001-Q001, or a marker closely linked thereto.

In certain embodiments of the methods described herein, the marker associated with high protein seeds is selected from the group consisting of an A at marker locus S20007K-001-Q001, a G at marker locus S20007N-001-Q001, a C at marker locus S20007R-001-Q001, a T at marker locus S20007T-001-Q001, an A at marker locus S20007W-001-Q001, a G at marker locus S200081-001-Q001, a C at marker locus S200083-001-Q001, a T at marker locus S200085-001-Q001, a C at marker locus S200086-001-Q001, a C at marker locus S200093-001-Q001, and a T at marker locus S20008A-001-Q001. In certain embodiments, the marker associated with high protein seeds is detected using a nucleic acid probe.

### BRIEF DESCRIPTION OF THE DRAWINGS AND THE SEQUENCE LISTING

The disclosure can be more fully understood from the following detailed description and the accompanying drawings and Sequence Listing, which form a part of this application.

FIG. 1 provides a sequence alignment of a portion of the Glyma.20g85100 coding region sequence in 3 high protein lines (SEQ ID NOs: 56 (pos: 5750-5878), 57 (pos: 5734-5862), and 58 (pos: 3937-4165)) and 3 elite low protein lines (SEQ ID NOs: 53 (pos: 5698-6147), 54 (pos: 5713-6162), and 55 (pos: 5698-6147)). A 321 bp insertion is present in the 3 low protein elite lines (SEQ ID NOs: 53, 54 and 55) and not in 3 high protein lines (SEQ ID NOs: 56, 57, and 58).

FIG. 2 provides a sequence alignment of a Glyma.20g85100 promoter region sequence in 3 high protein lines (SEQ ID NOs: 56 (pos: 2101-2250), 57 (pos: 2090-2239), and 58 (pos: 297-446)) and 3 elite low protein lines (SEQ ID NOs: 53 (pos: 2070-2219), 54 (pos: 2085-2234), and 55 (pos: 2070-2219)). The * shown in the alignment sits above an A/G SNP which can be used to track the high protein allele in a population.

The sequence descriptions (Table 1A and 1B) and sequence listing attached hereto comply with the rules governing nucleotide and amino acid sequence disclosures in patent applications as set forth in 37 C.F.R. §§ 1.831-1.835.

TABLE 1A
Sequence Listing Description - Markers

| | Wild- | Wild- | High | High | | |
|---|---|---|---|---|---|---|
| | Type | Type | Protein | Protein | Forward | Reverse |
| | Allele | probe | Allele | Probe | Primer | Primer |
| | SEQ ID | SEQ ID | SEQ ID | SEQ ID | SEQ ID | SEQ ID |
| Marker | NO: | NO: | NO: | NO: | NO: | NO: |
| S20007K- | 60 | 1 | 61 | 2 | 3 | 4 |
| 001-Q001 | | | | | | |
| S20007N- | 62 | 5 | 63 | 6 | 7 | 8 |
| 001-Q001 | | | | | | |
| S20007R- | 64 | 9 | 65 | 10 | 11 | 12 |
| 001-Q001 | | | | | | |
| S20007T- | 66 | 13 | 67 | 14 | 15 | 16 |
| 001-Q001 | | | | | | |
| S20007W- | 68 | 17 | 69 | 18 | 19 | 20 |
| 001-Q001 | | | | | | |
| S200081- | 70 | 21 | 71 | 22 | 23 | 24 |
| 001-Q001 | | | | | | |
| S200083- | 72 | 25 | 73 | 26 | 27 | 28 |
| 001-Q001 | | | | | | |
| S200085- | 74 | 29 | 75 | 30 | 31 | 32 |
| 001-Q001 | | | | | | |
| S200086- | 76 | 33 | 77 | 34 | 35 | 36 |
| 001-Q001 | | | | | | |
| S20008A- | 78 | 37 | 79 | 38 | 39 | 40 |
| 001-Q001 | | | | | | |
| S200093- | 80 | 41 | 81 | 42 | 43 | 44 |
| 001-Q001 | | | | | | |
| S200099- | | | | 45 | 46 | 47 |
| 00-Q001 | | | | | | |
| High protein | | | | | | |
| S200099- | | 48 | | | 49 | 50 |
| 00-Q001 | | | | | | |
| wild-type | | | | | | |

TABLE 1B
Sequence Listing Description - other sequences

| Sequence Name | SEQ ID NO: |
|---|---|
| Gm-CCT wildtype | 51 |
| Gm-CCT high protein variant | 52 |
| 93B86 20g85100 genomic | 53 |
| Williams82 20g85100 genomic | 54 |
| 93Y21 20g85100 genomic | 55 |
| PI 437.088A HP 20g85100 genomic | 56 |
| PI678444 HP 20g85100 | 57 |
| PI468916 HP 20g85100 genomic | 58 |
| high protein CCT allele deletion | 59 |

### DETAILED DESCRIPTION

Over time, the proportion of protein in seed of elite soybean varieties has declined as yield has steadily increased through breeding selections. The present disclosure provides methods and compositions for producing, detecting, and selecting soybean plants and seeds comprising at least one high protein CCT (CONSTANS, CO-like and TOC1) domain containing glyma.20g085100 variant (SEQ ID NO: 52) allele and introgressing the high protein CCT variant allele into soybean plants. The methods allow for the identification of soybean plants and seeds homozygous for the high protein allele and plants heterozygous for the high protein allele, which supports selections in earlier breeding stages of soybean breeding programs, such that plants with desirable high protein alleles are efficiently advanced to late-stage testing.

Accordingly, provided herein is a method for producing plants comprising a high protein CCT allele comprising isolating nucleic acids from a soybean plant or soybean germplasm population comprising a plurality of soybean plants, the soybean plants comprising a CCT gene and the soybean population comprising a high protein CCT allele of the CCT gene and a wild-type CCT allele of the CCT gene, assaying the one or more nucleic acids for the presence of the high protein CCT allele, assaying the one or more nucleic acids for the presence of the wild-type CCT allele, selecting from the plurality of soybean plants one or more soybean plants comprising two high protein CCT alleles or comprising one high protein CCT allele and one wild-type CCT allele, or a combination thereof. In certain embodiments, the one or more plants selected is homozygous for the high protein CCT allele. In certain embodiments, the method further comprises crossing the selected soybean plants with a second soybean plant, optionally comprising at least one high protein CCT allele, or self-pollinating the selected plants, to produce a plant having the high-protein CCT allele. In certain embodiments, the plant produced is homozygous for the high protein CCT allele. In certain embodiments, the method further comprises detecting in the one or more nucleic acids at least one marker locus associated with high protein seeds and/or the high protein CCT allele, suitable markers for use in the method are disclosed herein and include marker loci located within a chromosome interval flanked by and including marker locus S20007K-001-Q001 (e.g., the marker locus detected by the nucleotide probe of SEQ ID NO: 2) and marker locus S20008A-001-Q001 (e.g., the marker locus detected by the nucleotide probe of SEQ ID NO: 38).

As used herein “allele” refers to any of one or more alternative forms of a genetic sequence. In a diploid cell or organism, the two alleles of a given sequence typically occupy corresponding loci on a pair of homologous chromosomes. With regard to a SNP marker, allele refers to the specific nucleotide base present at that SNP locus in that individual plant. A “favorable allele” as used herein refers to the allele at a particular locus (a marker, a QTL, a gene etc.) that confers, or contributes to, an agronomically desirable phenotype, e.g., high protein seed, and that allows the identification of plants with that agronomically desirable phenotype. A favorable allele of a marker is a marker allele that segregates with the favorable phenotype. An “unfavorable allele” of a marker is a marker allele that segregates with the unfavorable plant phenotype, therefore providing the benefit of identifying plants that can be removed from a breeding program or planting.

As used herein, the term “crossing”, “crossed”, “cross” or the like refers to a sexual cross and involves the fusion of two haploid gametes via pollination to produce diploid progeny (e.g., cells, seeds, or plants). The term encompasses both the pollination of one plant by another and selfing (or self-pollination, e.g., when the pollen and ovule are from the same plant).

As used herein, the term “plant” includes plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like.

In certain embodiments of the methods described herein, the steps of assaying the one or more nucleic acids for the presence of the high protein CCT allele and the wild-type CCT allele occurs in the same reaction vessel. In certain embodiments, the steps of assaying the one or more nucleic acids for the presence of the high protein CCT allele and the wild-type CCT allele occurs simultaneously in the same reaction vessel. In certain embodiments, the steps of assaying the one or more nucleic acids for the presence of the high protein CCT allele and the wild-type CCT allele occurs sequentially in the same reaction vessel. In certain embodiments, the steps of assaying the one or more nucleic acids for the presence of the high protein CCT allele and the wild-type CCT allele occurs in separate reaction vessels.

The method for detecting the presence of the high protein CCT allele is not particularly limited and includes any method that can selectively differentiate between the high protein CCT allele and the wild-type CCT allele. In certain embodiments, assaying for the presence of the high protein CCT allele comprises detecting a nucleotide deletion in the CCT gene sequence (e.g., SEQ ID NO: 51). In certain embodiments, assaying for the presence of the high protein CCT allele comprises detecting a nucleotide deletion of at least 10, 25, 50, 75, 100, 125, 150, 175, 200, 225, 250, 275, 300, or 325 nucleotides in the CCT gene sequence. In certain embodiments, the at least 10, 25, 50, 75, 100, 125, 150, 175, 200, 225, 250, 275, 300, or 325 nucleotides in the CCT gene sequence are consecutive nucleotides in the CCT gene sequence. In certain embodiments, the high protein CCT allele comprises a nucleotide deletion of a nucleotide sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity SEQ ID NO: 59 in the CCT gene sequence, such that in certain embodiments assaying for the presence of the high protein CCT allele comprises detecting a nucleotide deletion of the sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity SEQ ID NO: 59 in the wild-type CCT gene sequence. In certain embodiments, the high protein CCT allele is detected using a nucleic acid probe that differentiates between the high protein and wild-type allele. In certain embodiments, the nucleotide probe selectively hybridizes to the nucleotides flanking the 5′ and 3′ ends of the nucleotide sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity SEQ ID NO: 59 in the wild-type CCT gene sequence. The number of flanking nucleotides recognized by the probe is not particularly limited as long as at least one 5′ flanking nucleotide and at least one 3′ flanking nucleotide is hybridized. In certain embodiments, the probe for detecting the high protein CCT allele comprises SEQ ID NO: 45.

The method for detecting the presence of the wild-type CCT allele is not particularly limited and includes any method that can selectively differentiate between the wild-type CCT allele and the high protein CCT allele. In certain embodiments, the presence of the wild-type CCT allele is determined by detecting the presence of the wild-type CCT allele having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to SEQ ID NO: 51. In certain embodiments, the presence of the wild-type CCT allele is determined by detecting the presence of a nucleotide sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to SEQ ID NO: 59 in the CCT gene sequence (e.g., SEQ ID NO: 51). In certain embodiments, the wild-type CCT allele is detected using a nucleic acid probe that selectively hybridizes the nucleotide sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to SEQ ID NO: 59 or a fragment thereof, such that the probe hybridizes to at least 1, 2, 3, 4, 5, 10, 20, 25, 30, 35, 40, 45, 50, 75, 100, 150, 200, 250, or 300 nucleotides of the nucleotide sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to SEQ ID NO: 59. In certain embodiments, the probe for detecting the wild-type CCT allele comprises SEQ ID NO: 48.

Also provided herein are methods for selecting plants in a segregating population comprising a high protein CCT allele comprising self-pollinating a first soybean plant or first soybean germplasm or crossing the first soybean plant or first soybean germplasm with a second soybean plant or second soybean germplasm to form a soybean population comprising a plurality of soybean plants or soybean germplasm, the soybean plants or soybean germplasm comprising a CCT gene and the soybean population comprising a high protein CCT allele of the CCT gene and a wild-type CCT allele of the CCT gene, isolating nucleic acids from the soybean plants or soybean germplasm of the population, assaying the one or more nucleic acids for the presence of the high protein CCT allele, assaying the one or more nucleic acids for the presence of the wild-type CCT allele, and selecting from the plurality of soybean plants or soybean germplasm one or more soybean plants or soybean germplasm comprising two high protein CCT alleles or comprising one high protein CCT allele and one wild-type CCT allele, or a combination thereof. In certain embodiments, the one or more soybean plants or soybean germplasm selected are homozygous for the high protein CCT allele. In certain embodiments, the method further comprises crossing the selected soybean plants or soybean germplasm with a different soybean plant, or self-pollinating the selected plants or germplasm, to produce a plant having the high protein CCT allele, optionally a plant homozygous for the high protein CCT allele. In certain embodiments, the method further comprises detecting in the one or more nucleic acids at least one marker locus associated with high protein seeds and/or the high protein CCT allele, suitable markers for use in the method are disclosed herein and include marker loci located within a chromosome interval flanked by and including marker locus S20007K-001-Q001 and marker locus S20008A-001-Q001. The method for assaying for the presence of the high protein CCT allele and the wild-type CCT allele may be any method known in the art that can selectively differentiate between the high protein CCT allele and the wild-type CCT allele, such as the methods of detection described herein. The assay steps can be performed in the same reaction vessel, either simultaneously or sequentially, or in different reaction vessels.

As used herein, the term “germplasm” refers to genetic material of or from an individual (e.g., a plant), a group of individuals (e.g., a plant line, variety or family), or a clone derived from a line, variety, species, or culture, or more generally, all individuals within a species or for several species (e.g., maize germplasm collection or Andean germplasm collection). The germplasm can be part of an organism, cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells, that can be cultured into a whole plant.

Further provided are methods for introgressing a high protein CCT domain containing variant sequence into a soybean plant or soybean germplasm comprising crossing a first soybean plant or first soybean germplasm with a second soybean plant or second soybean germplasm to form a soybean plant or soybean germplasm population, wherein the first soybean plant or soybean germplasm or the second soybean plant or germplasm comprises the high protein CCT domain containing variant sequence, isolating nucleic acids from the soybean plants or soybean germplasm of the population, assaying the one or more nucleic acids for the presence of a high protein CCT allele, assaying the one or more nucleic acids for the presence of a wild-type CCT allele, and selecting from the plurality of soybean plants or soybean germplasm one or more soybean plants or soybean germplasm comprising at least one high protein CCT allele. In certain embodiments, the one or more soybean plants or soybean germplasm selected are homozygous for the high protein CCT allele. In certain embodiments, the method further comprises crossing the selected soybean plants or soybean germplasm with a different soybean plant, or self-pollinating the selected plants or germplasm, to produce a plant having the high protein CCT allele, optionally a plant homozygous for the high protein CCT allele. In certain embodiments, the method further comprises detecting in the one or more nucleic acids at least one marker locus associated with high protein seeds and/or the high protein CCT allele, suitable markers for use in the method are disclosed herein and include marker loci located within a chromosome interval flanked by and including marker locus S20007K-001-Q001 and marker locus S20008A-001-Q001. The method for assaying for the presence of the high protein CCT allele and the wild-type CCT allele may be any method known in the art that can selectively differentiate between the high protein CCT allele and the wild-type CCT allele, such as the methods of detection described herein. The assay steps can be performed in the same reaction vessel, either simultaneously or sequentially, or in different reaction vessels.

“Introgressing”, “introgression” and the like, as used herein, refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be detected by a marker that is associated with a phenotype, e.g., at a QTL, a transgene, or the like. Offspring comprising the desired allele may be repeatedly backcrossed to a line having a desired genetic background and selected for the desired allele, to result in the allele becoming fixed in a selected genetic background. The process of “introgressing” is often referred to as “backcrossing” when the process is repeated two or more times.

Also provided are methods and compositions for producing, detecting, and selecting soybean plants producing seeds having a high protein content including breeding methods for introgressing high protein alleles into soybean plants using markers, e.g., single-nucleotide polymorphism (SNP) markers, linked to or associated with high protein CCT variant (SEQ ID NO: 52), in soybean.

In certain embodiments, the method comprises isolating nucleic acids from a soybean plant or soybean germplasm population, the population comprising a plurality of soybean plants; and detecting in the isolated nucleic acids at least one (e.g., 1, 2, 3, 4, 5, 6, 7, 8, 9, 10 or more) marker locus linked to or associated with high protein seeds located within a chromosomal interval flanked by and including marker locus S20007K-001-Q001 and marker locus S20008A-001-Q001, wherein the chromosomal interval comprises at least one of an A at marker locus S20007K-001-Q001, a G at marker locus S20007N-001-Q001, a C at marker locus S20007R-001-Q001, a T at marker locus S20007T-001-Q001, an A at marker locus S20007W-001-Q001, an M at marker locus S200099-00-Q001, a G at marker locus S200081-001-Q001, a C at marker locus S200083-001-Q001, a T at marker locus S200085-001-Q001, a C at marker locus S200086-001-Q001, a C at marker locus S200093-001-QOO1, and a T at marker locus S20008A-001-Q001. In certain embodiments, the method further comprises selecting plants comprising the detected maker locus linked to or associated with high protein seeds, e.g., selecting plants having a favorable allele for high protein seeds. In certain embodiments, the method further comprises crossing the selected plant with a second plant to produce progeny, wherein the progeny comprise the marker locus linked to or associated with high protein seed. In certain embodiments, the second soybean plant is an elite soybean strain. Also contemplated herein are embodiments in which plants are selected that do not comprising the maker locus linked to or associated with high protein seeds, e.g., selecting plants having an unfavorable allele for high protein seeds. In certain embodiments, these selected seeds are removed from the breeding program.

In certain embodiments, the at least one marker locus linked to or associated with high protein seeds comprises a marker locus linked to or associated with the high protein CCT domain containing glyma.20g085100 variant (SEQ ID NO: 52).

In certain embodiments, the marker locus is located within a chromosomal interval flanked by and including marker locus S20007N-001-Q001 and marker locus S200093-001-Q001, wherein the chromosomal interval comprises at least one of a G at marker locus S20007N-001-Q001, a C at marker locus S20007R-001-Q001, a T at marker locus S20007T-001-Q001, an A at marker locus 520007W-001-Q001, an M at marker locus S200099-00-Q001, a G at marker locus S200081-001-Q001, a C at marker locus S200083-001-Q001, a T at marker locus S200085-001-Q001, a C at marker locus S200086-001-Q001, and a C at marker locus S200093-001-Q001.

In certain embodiments, the marker locus is located within a chromosomal interval flanked by and including marker locus S20007R-001-Q001 and marker locus S200086-001-Q001, wherein the chromosomal interval comprises at least one of a C at marker locus S20007R-001-Q001, a T at marker locus S20007T-001-Q001, an A at marker locus S20007W-001-Q001, an M at marker locus S200099-00-Q001, a G at marker locus S200081-001-Q001, a C at marker locus 5200083-001-Q001, a T at marker locus 5200085-001-Q001, and a C at marker locus 5200086-001-Q001.

In certain embodiments, the marker locus is located within a chromosomal interval flanked by and including marker locus 520007T-001-Q001 and marker locus S200085-001-Q001, wherein the chromosomal interval comprises at least one of a T at marker locus S20007T-001-Q001, an A at marker locus S20007W-001-Q001, an M at marker locus S200099-00-Q001, a G at marker locus S200081-001-Q001, a C at marker locus S200083-001-Q001, and a T at marker locus S200085-001-Q001.

In certain embodiments, the marker locus is located within a chromosomal interval flanked by and including marker locus S20007W-001-Q001 and marker locus S200083-001-QOO1, wherein the chromosomal interval comprises at least one of an A at marker locus S20007W-001-Q001, an M at marker locus S200099-00-Q001, a G at marker locus S200081-001-Q001, and a C at marker locus S200083-001-Q001.

In certain embodiments, the at least one marker locus linked to or associated with high protein seed comprises a marker locus within about 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 110 kb, 120 kb, 130 kb, 140 kb, 150 kb, 160 kb, 170 kb, 180 kb, 190 kb, or about 200 kb of a marker locus selected from the group consisting of S20007K-001-QOO1, S20007N-001-QOO1, S20007R-001-QOO1, S20007T-001-QOO1, S20007W-001-Q001, S200099-00-Q001, S200081-001-QOO1, S200083-001-QOO1, S200085-001-Q001, S200086-001-QOO1, S200093-001-QOO1, and S20008A-001-QOO1.

In certain embodiments, detecting comprises detecting at least one marker locus selected from the consisting of S20007K-001-Q001, S20007N-001-Q001, S20007R-001-Q001, S20007T-001-Q001, S20007W-001-QOO1, S200099-00-Q001, S200081-001-QOO1, S200083-001-QOO1, S200085-001-Q001, S200086-001-Q001, S200093-001-Q001, and S20008A-001-Q001, or a maker closely linked thereto.

As used herein, “closely linked” means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., high seed protein content). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9% 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

In certain embodiments, the marker linked to or associated with high protein seed is within 50 cM, 40 cM, 30 cM, 25 cM, 20 cM, 15 cM, 10 cM, 9 cM, 8 cM, 7 cM, 6 cM, 5 cM, 4 cM, 3 cM, 2 cM, 1 cM of one or more markers selected from the group consisting of S20007K-001-Q001, S20007N-001-Q001, S20007R-001-Q001, S20007T-001-Q001, S20007W-001-Q001, S200099-00-Q001, S200081-001-Q001, S200083-001-Q001, S200085-001-Q001, S200086-001-Q001, S200093-001-Q001, and S20008A-001-Q001.

A common measure of linkage is the frequency with which traits cosegregate. This can be expressed as a percentage of cosegregation (recombination frequency) or in centiMorgans (cM). The cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency. Marker loci are themselves traits and can be assessed according to standard linkage analysis by tracking the marker loci during segregation. Thus, one cM is equal to a 1% chance that a marker locus will be separated from another locus, due to crossing over in a single generation.

As used herein, the term “associated with” in connection with a relationship between a marker locus and a phenotype refers to a statistically significant dependence of marker frequency with respect to a quantitative scale or qualitative gradation of the phenotype. Thus, an allele of a marker is associated with a trait of interest when the allele of the marker locus and the trait phenotypes are found together in the progeny of an organism more often than if the marker genotypes and trait phenotypes segregated separately.

When a trait is stated to be linked to a given marker it will be understood that the actual DNA segment whose sequence affects the trait generally co-segregates with the marker.

As used herein, “chromosome interval”, “chromosomal interval” and the like refers to a chromosome segment defined by specific flanking marker loci. The term “chromosome segment” designates a contiguous linear span of genomic DNA that resides in planta on a single chromosome.

As used herein, “marker” or “molecular marker” or “marker locus” denotes a nucleic acid or amino acid sequence that is sufficiently unique to characterize a specific locus on the genome. Any detectable polymorphic trait can be used as a marker so long as it is inherited differentially and exhibits linkage disequilibrium with a phenotypic trait of interest. Examples of markers for use in the methods described herein, include, but are not limited to, simple sequence repeats (SSRs), single nucleotide polymorphisms (SNPs), restriction fragment length polymorphisms (RFLPs), and indels. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

As used herein, a “single nucleotide polymorphism (SNP)” refers to a DNA sequence variation occurring when a single nucleotide—A, T, C—or G in the genome (or other shared sequence) differs between members of a biological species or paired chromosomes in an individual.

The term “indel” refers to an insertion or deletion, wherein one line may be referred to as having an inserted nucleotide or piece of DNA relative to a second line, or the second line may be referred to as having a deleted nucleotide or piece of DNA relative to the first line.

In certain embodiments, at least two marker loci (e.g., 2, 3, 4, 5, 6, 7, 8, 9, 10) linked to or associated with high protein seed (e.g., marker loci linked to or associated with the high protein CCT domain containing glyma.20g085100 variant) are detected. In certain embodiments, the at least two marker loci comprise a haplotype that is associated with increased seed protein.

As used herein, “haplotype” refers to a combination of particular alleles present within a particular plant's genome at two or more linked marker loci, for instance at two or more loci on a particular linkage group.

In certain embodiments, the molecular markers or marker loci are detected using a suitable amplification-based detection method, such as, for example, PCR, RT-PCR, and LCR. PCR, RT-PCR, and LCR are in particularly broad use as amplification and amplification-detection methods for amplifying nucleic acids of interest (e.g., those comprising marker loci), facilitating detection of the markers. Such nucleic acid amplification techniques can be applied to amplify and/or detect nucleic acids of interest, such as nucleic acids comprising marker loci. In these types of methods, nucleic acid primers are typically hybridized to the conserved regions flanking the polymorphic marker region. In certain methods, nucleic acid probes that bind to the amplified region are also employed. In general, synthetic methods for making oligonucleotides, including primers and probes, are well known in the art. The primers and probes for use in the methods described herein is not particularly limited and may be designed using methods and/or software known in the art, such as, for example, LASERGENE© or Primer3. It is not intended that the primers be limited to generating an amplicon of any particular size. For example, the primers used to amplify the marker loci and alleles herein are not limited to amplifying the entire region of the relevant locus. In some embodiments, marker amplification produces an amplicon at least 20 nucleotides in length, or alternatively, at least 50 nucleotides in length, or alternatively, at least 100 nucleotides in length, or alternatively, at least 200 nucleotides in length.

Non-limiting examples of polynucleotide primers useful for detecting the marker loci provided herein are provided in Table 2 and 3 and include, for example, SEQ ID NOS: 3, 4, 7, 8, 11, 12, 15, 16, 19, 20, 23, 24, 27, 28, 31, 32, 35, 36, 39, 40, 46, 47, 49, and/or 50 or variants or fragments thereof.

Non-limiting examples of polynucleotide probes useful for detecting the marker loci associated provided herein include, for example, SEQ ID NO: 1, 5, 9, 13, 17, 21, 25, 29, 33, 37, 41, or 45 or any combination thereof.

In certain embodiments, probes used in detecting the markers described herein will possess a detectable label. Any suitable label can be used with a probe. Detectable labels suitable for use with nucleic acid probes include, for example, any composition detectable by spectroscopic, radioisotopic, photochemical, biochemical, immunochemical, electrical, optical, or chemical means. Useful labels include biotin for staining with labeled streptavidin conjugate, magnetic beads, fluorescent dyes, radiolabels, enzymes, and colorimetric labels. Other labels include ligands, which bind to antibodies labeled with fluorophores, chemiluminescent agents, and enzymes. Detectable labels may also include reporter-quencher pairs, such as those employed in Molecular Beacon and TaqMan™ probes. Generally, whether the quencher is fluorescent or simply releases the transferred energy from the reporter by non-radiative decay, the absorption band of the quencher should at least substantially overlap the fluorescent emission band of the reporter to optimize the quenching. Non-fluorescent quenchers or dark quenchers typically function by absorbing energy from excited reporters, but do not release the energy radiatively. Selection of appropriate reporter-quencher pairs for particular probes may be undertaken in accordance with known techniques.

Further, it will be appreciated that amplification is not a requirement for marker detection—for example, one can directly detect unamplified genomic DNA simply by performing a Southern blot on a sample of genomic DNA. Procedures for performing Southern blotting, amplification e.g., (PCR, LCR, or the like), and many other nucleic acid detection methods are well established.

Real-time amplification assays, including MB or TaqMan™ based assays, are especially useful for detecting SNP alleles. In such cases, probes are typically designed to bind to the amplicon region that includes the SNP locus, with one allele-specific probe being designed for each possible SNP allele. For instance, if there are two known SNP alleles for a particular SNP locus, “A” or “C,” then one probe is designed with an “A” at the SNP position, while a separate probe is designed with a “C” at the SNP position. While the probes are typically identical to one another other than at the SNP position, they need not be. For instance, the two allele-specific probes could be shifted upstream or downstream relative to one another by one or more bases. However, if the probes are not otherwise identical, they should be designed such that they bind with approximately equal efficiencies, which can be accomplished by designing under a strict set of parameters that restrict the chemical properties of the probes. Further, a different detectable label, for instance a different reporter-quencher pair, is typically employed on each different allele-specific probe to permit differential detection of each probe. In certain examples, each allele-specific probe for a certain SNP locus is 11-20 nucleotides in length, dual-labeled with a florescence quencher at the 3′ end and either the 6-FAM (6-carboxyfluorescein) or VIC (4,7,2′-trichloro-7′-phenyl-6-carboxyfluorescein) fluorophore at the 5′ end.

To effectuate SNP allele detection, a real-time PCR reaction can be performed using primers that amplify the region including the SNP locus, the reaction being performed in the presence of all allele-specific probes for the given SNP locus. By then detecting signal for each detectable label employed and determining which detectable label(s) demonstrated an increased signal, a determination can be made of which allele-specific probe(s) bound to the amplicon and, thus, which SNP allele(s) the amplicon possessed. For instance, when 6-FAM- and VIC-labeled probes are employed, the distinct emission wavelengths of 6-FAM (518 nm) and VIC (554 nm) can be captured. A sample that is homozygous for one allele will have fluorescence from only the respective 6-FAM or VIC fluorophore, while a sample that is heterozygous at the analyzed locus will have both 6-FAM and VIC fluorescence.

Other techniques for detecting SNPs can also be employed, such as allele specific hybridization (ASH). ASH technology is based on the stable annealing of a short, single-stranded, oligonucleotide probe to a completely complementary single-stranded target nucleic acid. Detection is via an isotopic or non-isotopic label attached to the probe. For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization.

In certain embodiments, the markers described herein are detected by genotyping. Several methods are available for SNP genotyping, including but not limited to, hybridization, primer extension, oligonucleotide ligation, nuclease cleavage, minisequencing, and coded spheres. The KASPar® and Illumina® Detection Systems are additional examples of commercially available marker detection systems. KASPar® is a homogeneous fluorescent genotyping system which utilizes allele specific hybridization and a unique form of allele specific PCR (primer extension) to identify genetic markers (e.g., a particular SNP marker lined to or associated with high soybean seed protein content). Illumina® detection systems utilize similar technology in a fixed platform format. The fixed platform utilizes a physical plate that can be created with up to 384 markers. The Illumina® system is created with a single set of markers that cannot be changed and utilizes dyes to indicate marker detection.

These systems and methods represent a wide variety of available detection methods which can be utilized to detect the markers described herein (e.g., marker loci linked to or associated with high seed protein content) but any other suitable method could also be used.

Further provided herein are methods for producing a soybean plant or soybean germplasm having increased seed protein content and methods for introgressing the high protein CCT domain containing glyma.20g085100 variant comprising crossing a crossing a first soybean plant or first soybean germplasm with a second soybean plant or second soybean germplasm to form a soybean plant or soybean germplasm population, isolating nucleic acids from the soybean plants or soybean germplasm of the population, detecting in the nucleic acids at least one marker locus associated with high protein seeds located within a chromosome interval flanked by and including marker locus S20007K-001-Q001 and marker locus S20008A-001-Q001, wherein the chromosomal interval comprises at least one of an A at marker locus S20007K-001-Q001, a G at marker locus S20007N-001-Q001, a C at marker locus S20007R-001-Q001, a T at marker locus S20007T-001-Q001, an A at marker locus S20007W-001-Q001, an M at marker locus S200099-00-Q001, a G at marker locus S200081-001-Q001, a C at marker locus S200083-001-Q001, a T at marker locus S200085-001-Q001, a C at marker locus S200086-001-Q001, a C at marker locus S200093-001-Q001, and a T at marker locus S20008A-001-Q001; and selecting, if present, one or more soybean plants or soybean germplasm of the population comprising the detected marker locus. In certain embodiments, the chromosomal interval and/or marker locus is any interval or marker described herein.

In certain embodiments of the methods described herein, the first soybean plant or soybean germplasm, the second soybean plant or soybean germplasm, or both the first and second soybean plant or soybean germplasm are elite soybean lines. In certain embodiments of the methods described herein, the first soybean plant or soybean germplasm is an exotic soybean line.

As used herein, and “elite line” is an agronomically superior line that has resulted from many cycles of breeding and selection for superior agronomic performance. Numerous elite lines are available and known to those of skill in the art of soybean breeding. As used herein, an “exotic soybean line” is a strain or germplasm derived from a soybean not belonging to an available elite soybean line or strain of germplasm. In the context of a cross between two soybean plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

In certain embodiments of the methods described herein, plants producing high protein seeds heterozygous or homozygous for the high protein CCT allele and/or comprising at least one marker described herein (e.g., high protein seed markers) comprise a protein content increase in the seed of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 and less than 3.0, 2.9, 2.8, 2.7, 2.6, 2.5, 2.4, 2.3, 2.2, 2.1, 2.0, 1.9, 1.8, 1.7, 1.6, or 1.5 percentage points by weight compared with a wild-type soybean seed (and plant producing the seed) not comprising the marker locus or high protein CCT allele. In certain embodiments, plants producing high protein seeds comprise seeds having a protein content of at least 30.0%, 30.5%, 31.0%, 31.5%, 32.0%, 32.5%, 33.0%, 33.5%, 34.0%, 34.5%, 35.0%, 35.5%, 36.0%, 36.5%, 37.0%, 37.5%, 38.0%, 38.5%, 39.0%, 39.5%, 40.0%, 40.5%, 41.0%, 41.5% or 42.0% (percentage points by weight) and less than 55%, 54%, 53%, 52%, 51%, 50%, 49%, 48%, 47%, 46%, 45% or 44% (percentage points by weight).

In certain embodiments of the methods described herein, the first soybean plant or germplasm and the second soybean plant or germplasm differ in seed protein content. In certain embodiments of the methods described herein, the first soybean plant or germplasm has at least about a 1, 1.5, 2, 2.5, 3, 3.5, 4, 5, 10, or 15 and less than 20, 15, 10, 9, 8, 7, 6, or 5 percentage point increase in seed protein measured on a dry weight basis, as compared to the second soybean plant or germplasm. In certain embodiments of the methods described herein, the second soybean plant or germplasm has at least about a 1, 1.5, 2, 2.5, 3, 3.5, 4, 5, 10, or 15 and less than 20, 15, 10, 9, 8, 7, 6, or 5 percentage point increase in seed protein measured on a dry weight basis, as compared to the first soybean plant or germplasm.

In certain embodiments of the methods described herein, the selected plant comprising the high protein CCT allele and/or the detected marker locus has at least about a 1, 1.5, 2, 2.5, 3, 3.5, 4, 5, 10, or 15 and less than 20, 15, 10, 9, 8, 7, 6, or 5 percentage point increase in seed protein measured on a dry weight basis, as compared to the second soybean plant or germplasm. In certain embodiments, selected plant comprising the high protein CCT allele and/or the detected marker locus has at least about a 1, 1.5, 2, 2.5, 3, 3.5, 4, 5, 10, or 15 and less than 20, 15, 10, 9, 8, 7, 6, or 5 percentage point increase in seed protein measured on a dry weight basis, as compared to the first soybean plant or germplasm.

As used herein, “percent increase” refers to a change or difference expressed as a fraction of the control value, e.g. {[modified/transgenic/test value (%)−control value (%)]/control value (%)}×100%=percent change, or {[value obtained in a first location (%)−value obtained in second location (%)]/value in the second location (%)}×100=percent change.

In certain embodiments, the selected soybean plant or germplasm comprising the high protein CCT allele and/or the detected marker locus is subject to further breeding, including, but not limited to, additional crosses with other lines, hybrids, backcrossing, or self-crossing. In certain embodiments, the selected soybean plant or germplasm comprising the detected marker locus is backcrossed to the parent line (e.g., first soybean plant or germplasm or second soybean plant or germplasm) to produce a line of soybean plants that has high seed protein content and optionally also has other desirable traits from one or more other soybean lines.

In certain embodiments of the methods described herein, the method further comprises measuring the protein content in the seed of the selected plant or a progeny plant thereof (e.g., backcross progeny). The method for determining seed protein content is not particularly limited and may be any method known in the art. In certain embodiments, the measuring of protein content is performed using non-destructive single-seed near-infrared analysis (SS-NIR) as described previously (Roesler et al Plant Physiol. 2016 878-893).

Soybean plants, seeds, tissue cultures, variants and mutants having improved seed protein content produced by the methods described herein are also provided. Soybean plants, seeds, tissue cultures, variants and mutants comprising one or more of the marker loci, one or more of the favorable alleles, and/or one or more of the haplotypes and having improved seed protein content are provided. Also provided are isolated nucleic acids, kits, and systems useful for the identification and/or selection methods disclosed herein.

The following are examples of specific embodiments of some aspects of the invention. The examples are offered for illustrative purposes only and are not intended to limit the scope of the invention in any way.

### Example 1

This example demonstrates the development of markers to selectively identify the Glyma.20g85100 high protein gene.

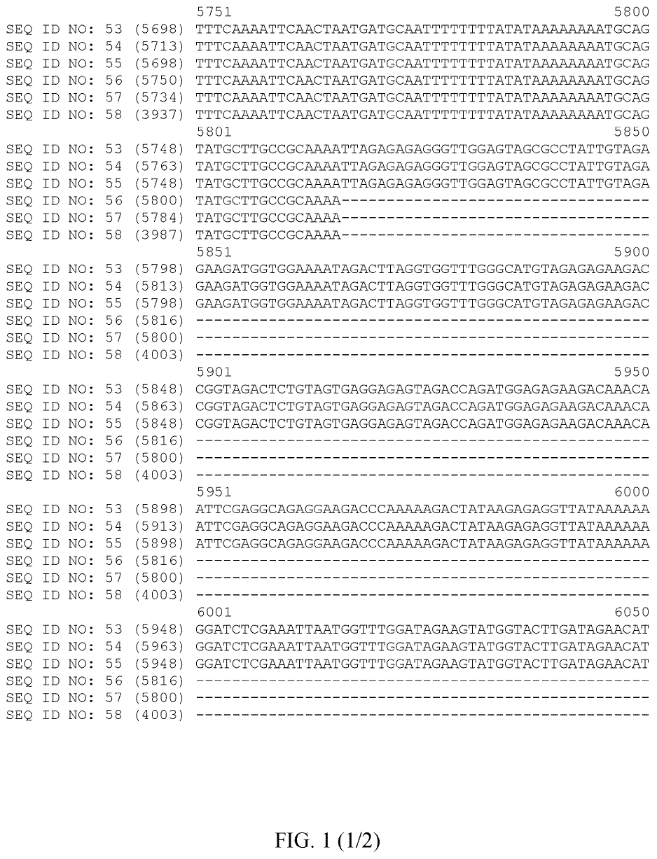

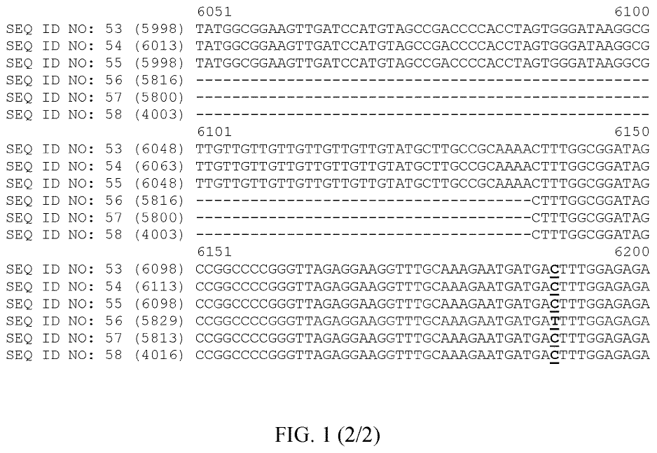

To selectively detect variants of a CCT domain containing gene (glyma.20g085100) on chromosome 20 containing a 321 bp insertion associated with high seed protein content a unique genotyping assay was developed that combines two separate assays -S200099-00-Q001. The first assay M (mutant-S200099-00-Q001 High protein from Table 1) detects the deletion (FAM) while the W (wildtype-S200099-00-Q001 wild-type from Table 1) assay (VIC), detects the wild type or insertion. Together these two assays in one well of a genotyping PCR reaction (such as the TaqMan assay described here) were used as a co-dominant marker to discriminate the high protein and low protein alleles in all zygocity states (FIG. 1). This assay is effective for foreground selection in the marker assisted backcross breeding as well as in trait purity applications.

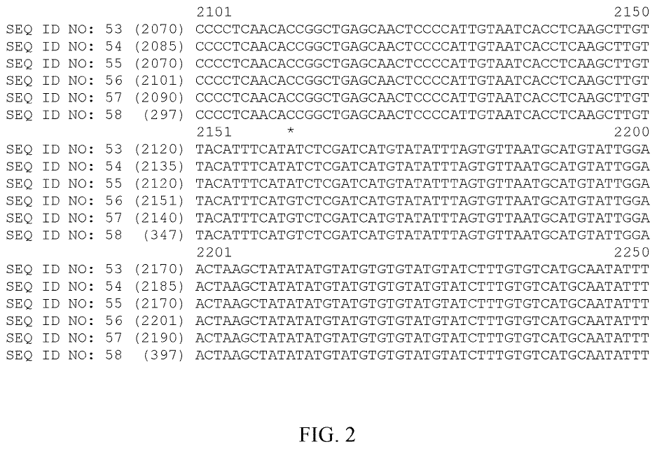

In addition, other SNPs were detected between high protein lines and low protein elite lines at promoter region of Glyma.20g085100 (FIG. 2). Gene specific marker targeting to promoter region can be developed to identify plants containing high protein allele in a backcross or F2 population.

### Example 2

This example demonstrates the development of flanking makers that identify the Glyma.20g85100 high protein gene.

Whole-genome shotgun sequence data for the donor line with high protein content P1678444 was generated (˜17X depth) using Illumina sequencing platform. The sequencing data was aligned to Williams 82 V2 reference genome and SNPs were discovered using standard SNP calling algorithm's (such as Bowtie 2) and compared against a proprietary SNP database of Corteva germplasm; This database contained 1475 soybean elite lines representing North America and Latin America. SNP's with very low minor allele frequency and highly specific to PI678444 were selected at 1 cM, 3 cM, 5 cM, 10 cM, 20 cM on either side of the glyma.20g085100 gene and converted them into genotyping assays. The minor allele frequencies (MAF) of the SNP's ranged from 0.12 to 20.99. Any methodology can be deployed to use this information, including but not limited to any one or more of sequencing or marker methods. In one example, sample tissue, including tissue from soybean leaves or seeds can be screened with the markers using a TAQMAN® PCR assay system (Life Technologies, Grand Island, NY, USA).

The TaqMan assays were developed as follow: Primers were designed using a software program. Probes were designed using Primer Express Software. 1.5 ul of the 1:100 DNA dilution was used in the assay mix. 18 uM of each probe, and 4 uM of each primer was combined to make each assay. 13.6 ul of the assay mix was combined with 1000 ul of lx BHQ Master Mix (Biosearch Technologies). A Meridian (Kbio) liquid handler dispensed 1.3 ul of the mix onto a 1536 plate containing ˜6 ng of dried DNA. The plate was sealed with a Phusion laser sealer and thermocycled using a Kbio Hydrocycler with the following conditions: 94C for 15 min, 40 cycles of 94C for 30 sec, 60C for 1 min. The excitation at wavelengths 485 (FAM) and 520 (VIC) was measured with a Pherastar plate reader. The values were normalized against ROX and plotted and scored on scatterplots utilizing the KRAKEN software.

### Example 3

This example demonstrates marker-assisted breeding for high protein soybean.

Phenotypic selection and recovery of high protein lines in each of the backcross progeny using single seed NIR to measure protein is complex as the environmental variation of single seed protein can be larger than the effect of QTL on seed protein. Marker assisted selection with SNPs in the Table 2, quickly allows selection of homozygous and heterozygous favorable alleles for early pre-selection in breeding saving phenotyping and field resources. This SNP panel is also useful for reducing linkage drag around the glyma.20g085100 gene and for rapid creation of elite high protein donors adapted to various maturity zones. The SNP markers identified here could also be useful, for example, for detecting soybean plants with high seed protein content, particularly useful for evaluating trait purity of commercial products as a quality check. The physical position of each SNP is provided in Table 2 based upon the JGI Glyma2 assembly (found online at phytozome-next.jgi.doe.gov/info/Gmax_Wm82_a2 v1). Any marker capable of detecting a polymorphism at one ofthese physical positions, or a marker associated, linked, or closely linked thereto, could also be useful, for example, for detecting and/or selecting soybean plants with high seed protein content. In some examples, the SNP allele present in the high protein parental line could be used as a favorable allele to detect or select plants with high protein content. In other examples, the SNP allele present in the low protein (high oil) parent line could be used as an unfavorable allele to detect or select plants with low protein content or high oil content. In Table 2, a+orientation (positive orientation) refers to the DNA strand that corresponds directly to the sequence of the RNA transcript which is translated to an amino acid sequence.

TABLE 2
Genomic features of the SNP markers

| | | | | | | | | | Probe |
|---|---|---|---|---|---|---|---|---|---|
| | | | | | | | | | Orientation |
| | | | | | | Donor- | | | (positive (+) |
| | | | | Physical | Genetic | High | RP/ | | or negative |
| Marker | Flanking | | Chrom | Position | Position | Protein | Wildtype | MAF | (−) strand) |
| S20007K- | 20 | cM | Chr20 | 1321134 | 20.17 | A (T in pos | G (C in pos | 9.46 | − |
| 001-Q001 | | | | | | 12 of SEQ | 11 of SEQ | | |
| | | | | | | ID NO: 2) | ID NO: 1) | | |
| S20007N- | 10 | cM | Chr20 | 2056362 | 25.84 | G (C in pos | A (T in pos | 20.99 | − |
| 001-Q001 | | | | | | 11 of SEQ | 14 of SEQ | | |
| | | | | | | ID NO: 6) | ID NO: 5) | | |
| S20007R- | 5 | cM | Chr20 | 4954137 | 35.5 | C (G in pos | T (A in pos | 0.81 | − |
| 001-Q001 | | | | | | 15 of SEQ | 15 of SEQ | | |
| | | | | | | ID NO: 10) | ID NO: 9) | | |
| S20007T- | 3 | cM | Chr20 | 12412587 | 37.5 | T (pos 10 | C (pos 10 | 0.16 | + |
| 001-Q001 | | | | | | of SEQ ID | of SEQ ID | | |
| | | | | | | NO: 14) | NO: 13) | | |
| S20007W- | 1 | cM | Chr20 | 30613510 | 39.5 | A (pos 10 | G (pos 10 | 0.23 | + |
| 001-Q001 | | | | | | of SEQ ID | of SEQ ID | | |
| | | | | | | NO: 18) | NO: 17) | | |
| S200099- | 0 | cM | Chr20 | 31778799 | 40.51 | M | W | | |
| 00-Q001 | | | | | | | | | |
| S200081- | 0 | cM | Chr20 | 31780445 | 40.51 | G (pos 10 | A (pos 13 | 0.32 | + |
| 001-Q001 | | | | | | of SEQ ID | of SEQ ID | | |
| | | | | | | NO: 20) | NO: 21) | | |
| S200083- | 1 | cM | Chr20 | 32867955 | 41.5 | C (G in pos | T (A in pos | 0.14 | − |
| 001-Q001 | | | | | | 8 of SEQ | 9 of SEQ ID | | |
| | | | | | | NO: ID 26) | NO: 25) | | |
| S200085- | 3 | cM | Chr20 | 33583343 | 43.51 | T (A in pos | C (G in pos | 0.12 | − |
| 001-Q001 | | | | | | 11 of SEQ | 11 of SEQ | | |
| | | | | | | NO: ID 30) | NO: ID 29) | | |
| S200086- | 5 | cM | Chr20 | 33889826 | 45.51 | C (G in pos | T (A in pos | 0.16 | − |
| 001-Q001 | | | | | | 10 of SEQ | 13 of SEQ | | |
| | | | | | | NO: ID 34) | NO: ID 33) | | |
| S200093- | 10 | cM | Chr20 | 34440896 | 50.15 | C (G in pos | T (A in pos | 0.38 | − |
| 001-Q001 | | | | | | 12 of SEQ | 16 of SEQ | | |
| | | | | | | NO: ID 42) | NO: ID 41) | | |
| S20008A- | 20 | cM | Chr20 | 35666258 | 60.5 | T (A in pos | C (G in pos | 3.26 | − |
| 001-Q001 | | | | | | 11 of SEQ | 12 of SEQ | | |
| | | | | | | NO: ID 37) | NO: ID 38) | | |

These SNP markers could also be used to determine a favorable or unfavorable haplotype. In certain examples, a favorable haplotype would include any combinations of S20007K-001-Q001 allele A, S20007N-001-Q00 allele G, S20007R-001-Q001 allele C, S20007T-001-Q001 allele T, S20007W-001-Q001 allele A, S200099-00-Q001 allele M, S200081-001-Q001 allele of G, S200083-001-Q001 allele of C, S200085-001-Q001 allele T, S200086-001-Q001 allele C, S200093-001-Q001 allele C, and S20008A-001-Q001 allele T (Table 2). In addition to the markers listed in Table 2, other closely linked markers could also be useful for detecting and/or selecting soybean plants with improved protein content. Further, chromosome intervals containing the markers provided herein could also be used, the chromosome interval on linkage group 20 flanked by and including S20007W-001-Q001-S200083-001-Q001, or an interval flanked by and including S20007T-001-Q001-S200085-001-Q001, or an interval flanked by and including S20007R-001-Q001-S200086-001-Q001 or an interval flanked by and including S20007N-001-Q001-S200093-001-Q001 or an interval flanked by and including S20007K-001-Q001-S20008A-001-Q001.

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1-20. (canceled)

21. A method selecting plants in a segregating population comprising a high protein CCT allele, the method comprising:
a. self-pollinating a first soybean plant or first soybean germplasm or crossing the first soybean plant or first soybean germplasm with a second soybean plant or second soybean germplasm to form a soybean population comprising a plurality of soybean plants or soybean germplasm, the soybean plants or soybean germplasm comprising a CCT gene and the soybean population comprising a high protein CCT allele of the CCT gene and a wild-type CCT allele of the CCT gene;
b. isolating nucleic acids from the soybean plants or soybean germplasm of the population;
c. assaying the one or more nucleic acids for the presence of the high protein CCT allele by detecting a nucleotide polymorphism in the CCT gene sequence having at least 95% identity to SEQ ID NO: 51;
d. assaying the one or more nucleic acids for the presence of the wild-type CCT allele having at least 95% identity to SEQ ID NO: 51; and
e. selecting from the plurality of soybean plants or soybean germplasm one or more soybean plants or soybean germplasm comprising two high protein CCT alleles or comprising one high protein CCT allele and one wild-type CCT allele, or a combination thereof.

22-24. (canceled)

25. The method of claim 21, wherein the nucleotide polymorphism comprises a deletion of a nucleic acid sequencing comprising at least 95% sequence identity to SEQ ID NO: 59.

26-27. (canceled)

28. The method of claim 25, wherein the deletion is detected using a nucleotide probe comprising SEQ ID NO: 45.

29. (canceled)

30. The method of claim 21, wherein the nucleotide polymorphism comprises a single nucleotide polymorphism (SNP) comprising a G at marker locus S200081-001-Q001.

31. (canceled)

32. The method of claim 21, wherein assaying for the presence of the wild-type CCT allele comprises detecting the presence of a nucleotide sequence having at least 95% sequence identity to SEQ ID NO: 59.

33. The method of claim 32, wherein the nucleotide sequence is detected using a nucleotide probe comprising SEQ ID NO: 48.

34. (canceled)

35. The method of claim 21, wherein assaying the one or more nucleic acids for the presence of the high protein CCT allele and the wild-type CCT allele occurs in the same reaction vessel.

36. The method of claim 21, wherein assaying the one or more nucleic acids for the presence of the high protein CCT allele and the wild-type CCT allele simultaneously.

37. The method of claim 21, wherein the method further comprises detecting in the one or more nucleic acids at least one marker locus associated with high protein seeds located within a chromosome interval flanked by and including marker locus S20007K-001-Q001 and marker locus S20008A-001-Q001.

38. (canceled)

39. The method of claim 37, wherein the marker associated with high protein seeds is selected from the group consisting of an A at marker locus S20007K-001-Q001, a G at marker locus S20007N-001-Q001, a C at marker locus S20007R-001-Q001, a T at marker locus S20007T-001-Q001, an A at marker locus S20007W-001-Q001, a G at marker locus S200081-001-Q001, a C at marker locus S200083-001-Q001, a T at marker locus S200085-001-Q001, a C at marker locus S200086-001-Q001, a C at marker locus S200093-001-Q001, and a T at marker locus S20008A-001-Q001.

40. (canceled)

41. A method for introgressing a high protein CCT domain containing variant sequence into a soybean plant or soybean germplasm, the method comprising:
a. crossing a first soybean plant or first soybean germplasm with a second soybean plant or second soybean germplasm to form a soybean plant or soybean germplasm population, wherein the first soybean plant or soybean germplasm or the second soybean plant or germplasm comprises the high protein CCT domain containing variant sequence;
b. isolating nucleic acids from the soybean plants or soybean germplasm of the population;
c. assaying the one or more nucleic acids for the presence of the high protein CCT allele by detecting a nucleotide polymorphism in the CCT gene sequence having at least 95% identity to SEQ ID NO: 51;
d. assaying the one or more nucleic acids for the presence of a wild-type CCT allele having at least 95% identity to SEQ ID NO: 51; and
e. selecting from the plurality of soybean plants or soybean germplasm one or more soybean plants or soybean germplasm comprising at least one high protein CCT allele.

42. The method of claim 41, wherein the one or more soybean plants or soybean germplasm selected in step (e) is homozygous for the high protein CCT allele.

43. The method of claim 41, wherein the nucleotide polymorphism comprises a deletion of a nucleic acid sequencing comprising at least 95% sequence identity to SEQ ID NO: 59.

44-45. (canceled)

46. The method of claim 43, wherein the nucleotide deletion is detected using a nucleotide probe comprising SEQ ID NO: 45.

47. (canceled)

48. The method of claim 41, wherein the polymorphism comprises a single nucleotide polymorphism (SNP), comprising a G at marker locus S200081-001-Q001.

49. (canceled)

50. The method of claim 41, wherein assaying for the presence of the wild-type CCT allele comprises detecting the presence of a nucleotide sequence having at least 95% sequence identity to SEQ ID NO: 59.

51-52. (canceled)

53. The method of claim 41, wherein assaying the one or more nucleic acids for the presence of the high protein CCT allele and the wild-type CCT allele occurs in the same reaction vessel.

54. The method of claim 41, wherein assaying the one or more nucleic acids for the presence of the high protein CCT allele and the wild-type CCT allele simultaneously.

55. The method of claim 41, wherein the method further comprises detecting in the one or more nucleic acids at least one marker locus associated with high protein seeds located within a chromosome interval flanked by and including marker locus S20007K-001-Q001 and marker locus S20008A-001-Q001.

56. (canceled)

57. The method of claim 55, wherein the marker associated with high protein seeds is selected from the group consisting of an A at marker locus S20007K-001-Q001, a G at marker locus S20007N-001-Q001, a C at marker locus S20007R-001-Q001, a T at marker locus S20007T-001-Q001, an A at marker locus S20007W-001-Q001, a G at marker locus S200081-001-Q001, a C at marker locus S200083-001-Q001, a T at marker locus S200085-001-Q001, a C at marker locus S200086-001-Q001, a C at marker locus S200093-001-Q001, and a T at marker locus S20008A-001-Q001.

58-76. (canceled)
